(19) United States
(12) Patent Application Publication
(10) Pub. No.: US 2024/0274244 A1
(43) Pub. Date: Aug. 15, 2024

(54) Systems and Methods for Generating Macromolecular Conformations Using Deep Learning

(71) Applicant: Georgia Tech Research Corporation, Atlanta, GA (US)

(72) Inventors: Yui Tik Pang, Atlanta, GA (US); James C. Gumbart, Atlanta, GA (US); Katie Kuo, Atlanta, GA (US)

(21) Appl. No.: 18/441,606

(22) Filed: Feb. 14, 2024

Related U.S. Application Data
(60) Provisional application No. 63/485,117, filed on Feb. 15, 2023.

(51) Int. Cl. G16C 20/50; G16C 20/70

(52) U.S. Cl. CPC G16C 20/50; G16C 20/70

(57) ABSTRACT

Systems and methods are disclosed for generating macromolecule conformations using deep learning and for training deep learning networks to generate macromolecular conformations. The system can, for example, generate the transition between two end states based on energy evaluation. Additionally, the system can output all-atom structures of the end states in different conformations. These macromolecules include proteins, RNA, and polymers, among others. The present disclosure also includes methods for training a deep-learning network to model macromolecular conformations according to a specific objective function. The training can include employing a generative adversarial neural network, for example, by using molecular dynamics or energy simulations to validate predicted states.

### CROSS REFERENCE TO RELATED APPLICATIONS

This patent application claims priority to, and the benefit of, U.S. provisional application, U.S. 63/485,117, filed on Feb. 15, 2023, which is hereby incorporated by reference herein in its entirety.

### TECHNICAL FIELD

This disclosure generally relates to methods of generating macromolecular conformations using neural networks.

### BACKGROUND

The nexus of pharmacology and machine learning has contributed to the drug discovery field of research. There is a bottle next in discovering drugs in an experimental lab, usually making incremental changes to known compounds and evaluating pharmacological activity. This process of drug discovery and evaluation of activity takes an incredible number of resources, which may result in a few useful new drugs.

The rise of machine learning models has come alongside their use in the chemical and biophysical research space. However, the techniques of machine learning, and in particular neural networks, which are the workhorse of in silico discovery, need to be particularly adapted for a particular application. In the case of in silico discovery of chemical compounds, often the class of compounds-solids, gas, small molecules, large molecules, solvation, etc.—dictate the underlying functional form of the system description and what approximations can be adapted for efficient use of computational resources.

A common hurdle in adapting a neural network model is collecting a sufficient amount of training data. In the chemical and biologics (e.g., proteins, antibodies, amino acids, etc.) space, the amount of verified training data is often limited. In some known solutions, training data of various chemical and biologic systems may be compiled to form a single training data set. This raises specific technical problems of training a model system for a coherent chemical or biologic environment.

### SUMMARY

Systems and methods are disclosed for generating macromolecule conformations using deep learning and for training deep learning networks to generate macromolecular conformations. The system can, for example, generate the transition between two end states based on energy evaluation. Additionally, the system outputs all-atom structures of the end states in different conformations. These macromolecules include proteins, RNA, and polymers, among others.

The present disclosure also includes methods for training a deep-learning network to model macromolecular conformations according to a specific objective function. The training can include employing a generative adversarial neural network, for example, by using molecular dynamics or energy calculations to validate predicted states.

In some aspects, the method described herein relates to generating a candidate macromolecule structure having one or more target properties, the method including: receiving a first input and a second input, wherein the first input and the second input are related to a first macromolecule state and a second macromolecule state, respectively; training a first neural network on respective energies of the first macromolecule state and the second macromolecule state and energies from an energy database; generating a plurality of macromolecule conformations in a low-dimension representation using the trained first neural network initiated with a set of random numbers; determining respective energy of each macromolecule conformation of the plurality of macromolecule conformations; selecting a set of candidate macromolecule conformations from the plurality of macromolecule conformations, wherein the set of candidate macromolecule conformations are selected based on one or more scores related to the first macromolecule state, the second macromolecule state, and the energy database; training a second neural network on macromolecule conformations of the first macromolecule state and the second macromolecule state and macromolecule conformations from a structure database, wherein the structure database includes conformations in all-atom representation and related low-dimension representation; determining, for each macromolecule conformation of the set of candidate macromolecule conformations, an all-atom representation from a low-dimension representation using the trained second neural network; determining a local minimum energy of each candidate macromolecule conformation of the set of candidate macromolecule conformations to generate a set of optimized candidate macromolecule conformations and related energies; adding the optimized candidate macromolecule conformations to the structure database and related energies to the energy database; outputting one or more conformations of the set of optimized candidate macromolecule conformations, wherein the one or more conformations of the set of optimized candidate macromolecule conformations are subsequently synthetized and/or simulated to evaluate for one or more target properties; and repeating the operations until a threshold associated the one or more target properties is reached or for a pre-determined number of iterations.

In some aspects, the first macromolecule state includes a plurality of macromolecule conformations, and the second macromolecule state includes a plurality of macromolecule conformations.

In some aspects, the first macromolecule state includes a single macromolecule conformation, and the second macromolecule state includes a single macromolecule conformation.

In some aspects, the method described herein further includes sampling the first macromolecule state and the second macromolecule state in equilibrium conditions using statistical simulations or physics-based simulations, wherein the sampled equilibrium conformations and related energies of the first macromolecule state and second macromolecule state are used for training the first neural network and the second neural network.

In some aspects, the method described herein relates to further includes determining respective energies of the first macromolecule state of the first input state and determining respective energies of the second macromolecule state of the second input state;

In some aspects, the method described herein relates to training the first neural network and the second neural network includes reinforcement learning operations.

In some aspects, the first neural network is a deep learning neural network.

In some aspects, the first neural network includes a generative adversarial network or diffusion model or one or more autoencoders.

In some aspects, the first neural network includes a generative adversarial network (GAN) configured, in an iterative manner, to generate a plausible low-energy pathway between the first macromolecule state and the second macromolecule state, wherein the GAN includes a discriminator configured to determine if the plausible low-energy pathway between the first macromolecule state and the second macromolecule state includes physically possible macromolecule conformations.

In some aspects, the method described herein relates to training of the generative adversarial network includes: (i) generating macromolecule conformations at a generator of the GAN; (ii) determining related energies of the generated macromolecule conformations at an energy predicting neural network; (iii) scoring related energies of the generated macromolecule conformations relative to the energy database at a discriminator of GAN; (iv) adjusting a weights of the generator of GAN based on the discriminator of GAN and the energy predicting neural network; (v) adjusting the weights of the discriminator of GAN to classify data from “real” dataset as real and data from generator of GAN as “fake”; (vi) passing, at least one generated macromolecule conformation, to an energy minimizer, wherein the energy minimizer is configured to determine a local minimum of an energy function describing the at least one generated macromolecule conformation, and wherein the energy minimizer is saved as part of the “real” dataset; (vii) adjusting the weights of the energy predicting neural network using the “real” dataset and its corresponding energy labels; and (viii) repeat above operations i-vii in an iterative manner.

In some aspects, the method described herein relates to determining, for each macromolecule conformation of the set of candidate macromolecule conformations, an all-atom representation from a low-dimension representation using the trained second neural network, includes: predicting a backbone orientation; scoring the predicted backbone orientation relative to the conformations of the structure database; predicting dihedral angles; scoring the predicted dihedral angles relative to the conformations of the structure database; adding the backbone orientation score and dihedral angles score to a loss function of the trained second neural network; determining a coordinates of all atoms relative to the scored backbone orientation and scored dihedral angles using the trained second neural network.

In some aspects, the method described herein relates to determining respective energies includes evaluating a force field energy function.

In some aspects, the first state input and the second state input include all-atom representations of macromolecule conformations.

In some aspects, the method described herein further includes converting the all-atom representations of macromolecule confirmation of the first state input and the second state input to low-dimension representations before training the first neural network.

In some aspects, the first state input and the second state input include low-dimension representations of macromolecule conformations.

In some aspects, the low-dimension representation is defined by dihedral angles, contact distances, or distances between selected macromolecule residues.

In some aspects, the low-dimension representation is defined by Ca pairwise distance space.

In some aspects, the selected set of candidate structures includes a number of candidate structures wherein the number is defined by a user input.

In some aspects, the set of candidate structures are equally spaced along a transition path between the first macromolecule state and the second macromolecule state.

In some aspects, the system described herein relates to generating a candidate macromolecule structure having one or more target properties, including: at least one processor; a memory operably coupled to the at least one processor, the memory having computer-executable instructions stored thereon, that when executed by the at least one processor, causes the processor to: receive a first input and a second input, wherein the first input and the second input are related to a first macromolecule state and a second macromolecule state, respectively; determine respective energies of the first macromolecule state and the second macromolecule state; train a first neural network on respective energies of the first macromolecule state and the second macromolecule state and energies from an energy database; generate a plurality of macromolecule conformations in a low-dimension representation using the trained first neural network initiated with a set of random numbers; determine respective energy of each macromolecule conformation of the plurality of macromolecule conformations; select a set of candidate macromolecule conformations from the plurality of macromolecule conformations, wherein the set of candidate macromolecule conformations are selected based on one or more scores related to the first macromolecule state, the second macromolecule state, and the energy database; train a second neural network on macromolecule conformations of the first macromolecule state and the second macromolecule state and macromolecule conformations from a structure database, wherein the structure database includes conformations in all-atom representation and related low-dimension representation; determine, for each macromolecule conformation of the set of candidate macromolecule conformations, an all-atom representation from a low-dimension representation using the trained second neural network; determine a local minimum energy of each candidate macromolecule conformation of the set of candidate macromolecule conformations to generate a set of optimized candidate macromolecule conformations and related energies; add the optimized candidate macromolecule conformations to the structure database and related energies to the energy database; output one or more of the set of optimized candidate macromolecule conformations, wherein the one or more conformations of the set of optimized candidate macromolecule conformations, wherein the one or more conformations of the set of optimized candidate macromolecule conformations are subsequently synthetized and/or simulated to evaluate for one or more target properties; and repeat the operations until a threshold associated the one or more target properties is reached or for a pre-determined number of iterations.

Additional advantages of the disclosed compositions and methods will be set forth in part in the description which follows, and in part will be obvious from the description. The advantages of the disclosed compositions and methods will be realized and attained by means of the elements and combinations particularly pointed out in the appended claims. It is to be understood that both the foregoing general description and the following detailed description are exemplary and explanatory only and are not restrictive of the disclosed compositions and methods, as claimed.

The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

FIG. 1 shows a schematic of an embodiment of the system architecture.

FIG. 2 shows a data flowchart of one embodiment of the disclosed method.

FIGS. 3A and 3B show example transition paths that have one transition state (FIG. 3A) and two transition states (FIG. 3B).

FIG. 4 shows a schematic of the Explorer network architecture.

FIG. 5 shows an example algorithm of the Explorer.

FIG. 6 shows an example algorithm of the WGAN critic and the Energy critic of the Explorer.

FIG. 7 shows an example algorithm of the Structure Builder.

FIG. 8 shows an example algorithm of a system training method.

FIGS. 9A-9B show example macromolecule docking site (FIG. 9A) and target property (FIG. 9B).

FIGS. 10A-10D show example macromolecules, such as antibodies (FIG. 10A) and pertactin (FIG. 10B), a model autotransporter passenger domain; a folded RNA molecule (FIG. 10C), and a polymer (FIG. 10D).

FIG. 11 shows an example of a polymer interacting with RNA.

FIG. 12A shows an example visualization of a folding pathway.

FIG. 12B shows an example of charged residues being brought together.

FIG. 13 shows an example of the system architecture.

FIG. 14 shows an example of the system architecture.

FIG. 15 shows a schematic of a general-purpose computer.

### DETAILED SPECIFICATION

To facilitate an understanding of the principles and features of various embodiments of the present invention, they are explained hereinafter with reference to their implementation in illustrative embodiments.

The disclosed system and method include a deep learning method that generates macromolecule conformations from limited example conformations. In one implementation, the method is configured to find a series of macromolecule conformations that connect two user-provided conformations, i.e. a transition path between two conformations (end-states). In some examples, the two conformations are protein conformations, which may be folded and unfolded or partially folded.

The described method enables generating transition paths in a few hours to a day, compared to previous practices, e.g. string methods, which require months of computational time.

A technical problem in this field is the sparse training data available, which the disclosed system and method address by training on the molecule of interest. Additionally, the method of training the system relies on reinforcement learning of the target macromolecule, which provides advantages of comparable environment of the molecular system. For example, training only on systems of molecules in a solvated environment provides continuity and a cohesive set of training data rather than relying on training data of various molecular environments, having a mixed dataset of molecules in solvent, vacuum, partial solvation, etc.

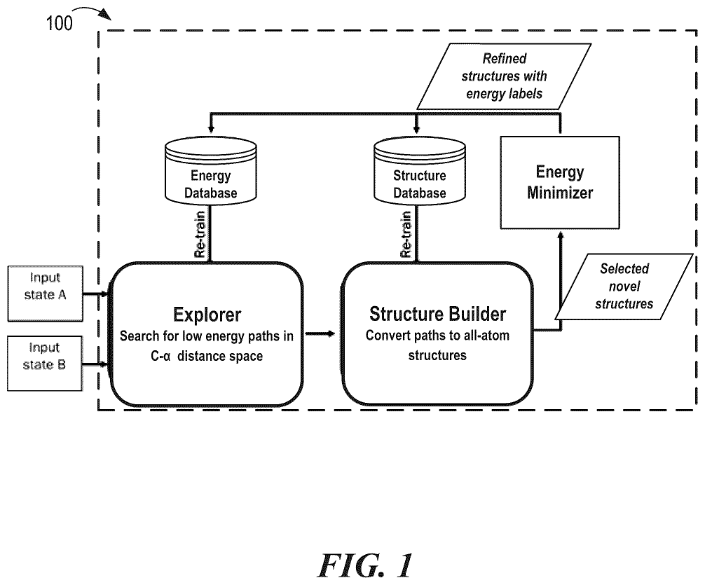

Referring now to FIG. 1A, the system 100 is shown. The system 100 is composed of two modules, the Explorer and Structure Builder. The Explorer constructs a transition path in a low-dimension space including conformations along the transition path, while the Structure Builder converts the conformations along the transition path from the low-dimension representation to all-atom representation.

The system 100 receives two end-state inputs, a first state input A and a second state input B, at the Explorer module. The Explorer module searches for conformations along low-energy paths from the first input state A to the second input state B, then the conformations included in the lowest-energy path are provided to the Structure Builder module, which converts the conformations along the lowest-energy path from low-dimensional representation to all-atom representations. The all-atom structures are passed to a molecular minimizer, which refines the all-atom structures and provides related ground-truth energy labels. The refined structures with energy labels are stored in a structure database and energy database, respectively.

In some implementations, the structure database includes a plurality of refined structures including the related low-dimension and all-atom representations.

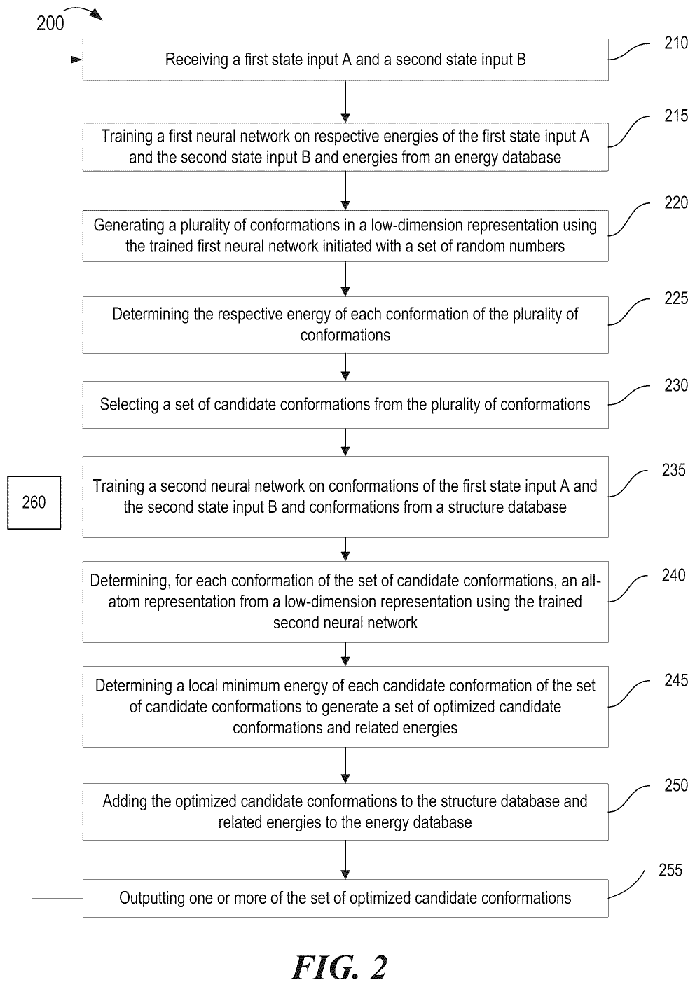

Referring now to FIG. 2, the exemplary method 200 includes receiving a first state input A and a second state input B 210, between which a low energy transition path is sought. The first state input A and the second state input B include a first macromolecule state and a second macromolecule state, respectively. Each state (e.g. macromolecule state), is described by the macromolecule conformation and related energy. The method further includes training a first neural network is on respective energies from the first macromolecule state, from the second macromolecule state, and from energies from an energy database 215, and generating a plurality of conformations in a low-dimension representation using the trained first neural network initiated with a set of random numbers. The method further includes determining the respective energy of each conformation of the generated plurality of conformations 225 and selecting a set of candidate conformations from the generated plurality of conformations 230, wherein the set of candidate macromolecule conformations are selected based on one or more scores related to the first macromolecule state, the second macromolecule state, and the energy database.

The method further includes training a second neural network on macromolecule conformations of the first macromolecule state and the second macromolecule state and macromolecule conformations from a structure database 235, wherein the structure database comprises conformations in all-atom representation and related low-dimension representation; determining, for each macromolecule conformation of the set of candidate macromolecule conformations, an all-atom representation from a low-dimension representation using the trained second neural network 240.

The method further includes determining a local minimum energy of each candidate macromolecule conformation of the set of candidate macromolecule conformations to generate a set of optimized candidate macromolecule conformations and related energies 245; adding the optimized candidate macromolecule conformations to the structure database and related energies to the energy database 250; outputting one or more conformations of the set of optimized candidate macromolecule conformations 255, wherein the one or more conformations of the set of optimized candidate macromolecule conformations are subsequently synthetized and/or simulated to evaluate for one or more target properties; and repeating the operations 260 until a threshold associated the one or more target properties is reached or for a pre-determined number of iterations.

In an aspect, the received input state includes a single or up to a few conformations, which is insufficient size of a training set for a neural network model. The received macromolecule conformations may be propagated in equilibrium conditions in a statistical simulation, such as Monte Carlo (MC) or a physics-based simulation, such as molecular dynamics (MD), or a statistical sampler, such as Monte Carlo (MC). It should be understood that the example simulation types are for example only and in no way limit the types of simulations that are contemplated by the description. It should be understood that any simulation or algorithm which provides a sufficient number of unique macromolecule conformations conform to the disclosure. In some aspects, a sufficient number of unique macromolecule conformations includes 100, 200, 300, 400 or more conformations. The output of the equilibrium simulation is a training set of macromolecule conformations in equilibrium at the respective states.

In other aspects, the received macromolecule conformations include a sufficient number of unique macromolecule conformations, which includes 100, 200, 300, 400 or more conformations.

In some aspects, the first and second state inputs comprise all-atom descriptions including point identification and cartesian coordinates of each atom. The first and second state inputs are converted from all-atom representation to a low-dimensional representation. In some aspects, a low-dimension representation is defined by dihedral angles, contact distances, or distances between selected macromolecule residues. The low-dimension representation may be, for example, Ca pairwise distance. In some aspects, the low-dimension representation may be in the form of a contact map, for example, when the macromolecule is a protein, a protein contact map may be used.

In some aspects, the first and second state inputs comprise macromolecule states comprising one or more conformations, but related energies are not provided. In cases where the input states do not contain related energies of each conformation, the related energies are determined using an indicated energy function. The energy function may be a force field energy function, a coarse-grained energy function, a pair wise potential energy function, a pseudopotential energy function, or any suitable energy function for the macromolecule states. The energy function may be provided as input or encoded with the other method functions, for example, the method includes determining respective energies of a macromolecule conformation using the indicated energy function.

In some aspects, training the first neural network includes reinforcement learning operations.

In some aspects, the first neural network is a deep learning neural network. In some aspects, the first neural network is a deep learning neural network including a generative adversarial neural network or diffusion model or one or more autoencoders. In some aspects, the first neural network is a deep learning neural network including a generative adversarial neural network configured, in an iterative manner, to generate a plausible low-energy pathway between the first macromolecule state and the second macromolecule state, wherein the GAN comprises a discriminator configured to determine if the plausible low-energy pathway between the first macromolecule state and the second macromolecule state comprises physically possible macromolecule conformations.

In some aspects, the selected set of candidate structures includes a number of candidate structures wherein the number is defined by a user input, and wherein the set of candidate structures are equally spaced along the transition path between the first macromolecule state and the second macromolecule state.

In some aspects, the training of the GAN includes: generating macromolecule conformations at a generator of the GAN; determining related energies of the generated macromolecule conformations at the energy predicting neural network; scoring related energies of the generated macromolecule conformations relative to the energy database at a discriminator of GAN; adjusting the weights of the generator of GAN based on the discriminator of GAN and the energy predicting neural network; adjusting the weights of the discriminator of GAN to classify data from “real” dataset as real and data from generator of GAN as “fake”; passing, at least one generated macromolecule conformation, to an energy minimizer, wherein the energy minimizer is configured to determine a local minimum of an energy function describing the at least one generated macromolecule conformation, wherein the local minimum of the energy function is related to the lowest energy of the conformation, and wherein the energy minimizer is saved as part of the “real” dataset; adjusting the weights of the energy predicting neural network using the “real” dataset and its corresponding energy labels; and repeat above operations in an iterative manner.

In some aspects, determining, for each macromolecule conformation of the set of candidate macromolecule conformations, an all-atom representation from a low-dimension representation using the trained second neural network, includes: predicting a backbone orientation; scoring the predicted backbone orientation relative to the conformations of the structure database; predicting dihedral angles; scoring the predicted dihedral angles relative to the conformations of the structure database; adding the backbone orientation score and dihedral angles score to a loss function of the trained second neural network; determining the coordinates of all atoms relative to the scored backbone orientation and scored dihedral angles using the trained second neural network.

In some aspects, the first neural network is related to the Explorer of system 100. The Explorer searches for low energy paths using the trained first neural network in the low-dimension representation. The low energy path relates to a path connecting the first macromolecular state to the second macromolecular state by the generated plurality of conformations, wherein the macromolecule states are defined by the respective conformations and energies. The low energy path is then defined by the plurality of conformations and the respective energies.

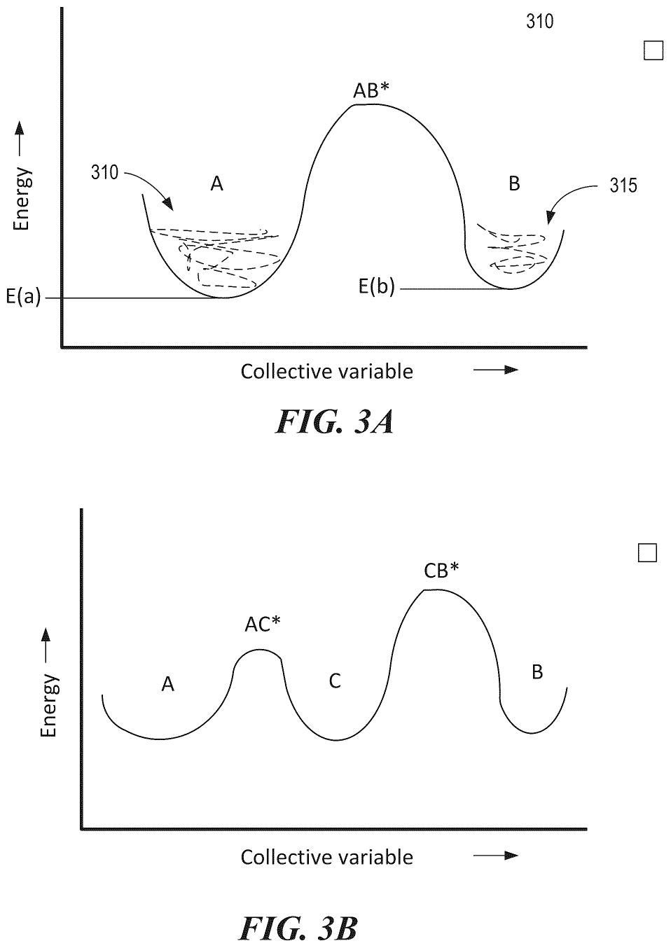

Referring now to FIG. 3A, it should be understood that a macromolecular state includes all conformations that exhibit the same energy upon conformation optimization. A macromolecular state (e.g., “A” or “B” of FIG. 3A) can include all conformations, whether sampled at equilibrium conditions or otherwise generated, that when optimized, exhibit the same energy (e.g., “E(a)” or “E(b)” of FIG. 3A). Conformations of a macromolecular state in equilibrium form an ensemble of conformations and related energies 310, 315 such that the exhibited energies are not the real energies (i.e. in equilibrium at room temperature in a molecular dynamics simulation). In one example, as a macromolecule changes conformations from a first state to a second state (e.g. Ah B), a high energy state may be reached between the first state and the second state, referred to as a transition state (e.g. AB*). In this example, any configuration on either side of the transition state will optimize to the first state having energy E(a) or second state having energy E(b). The sequence of macromolecular conformations between the first state and the second state and including the transition state make up the transition path. It should be understood that this is one description of an example transition path.

Referring now to FIG. 3B, in some examples, the transition path may contain an intermediate state, such as state C, separated from each of the first state and the second state by a first transition state AC* and a second transition state CB*.

The main task of Explorer is to construct a low energy path between the two end-states in the selected low-dimensional representation. In this implementation, an adapted progressive generative adversarial network (GAN) is used, such as StyleGAN, which generates the path in a progressive manner by first training the model to generate a few intermediate conformations (images) along the path then progressively increase the network complexity and the number of intermediate conformations (images) generated along the path.

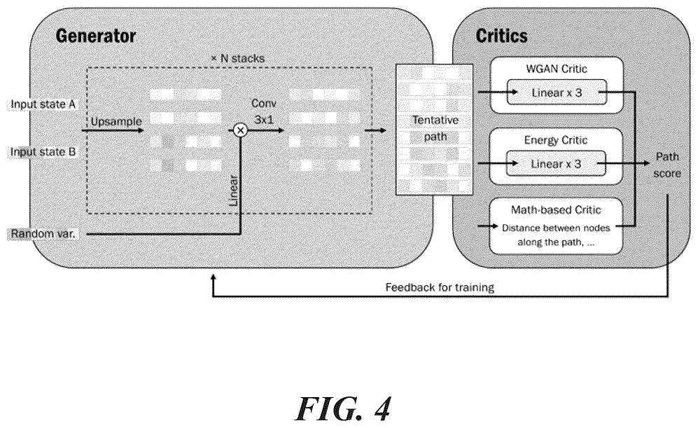

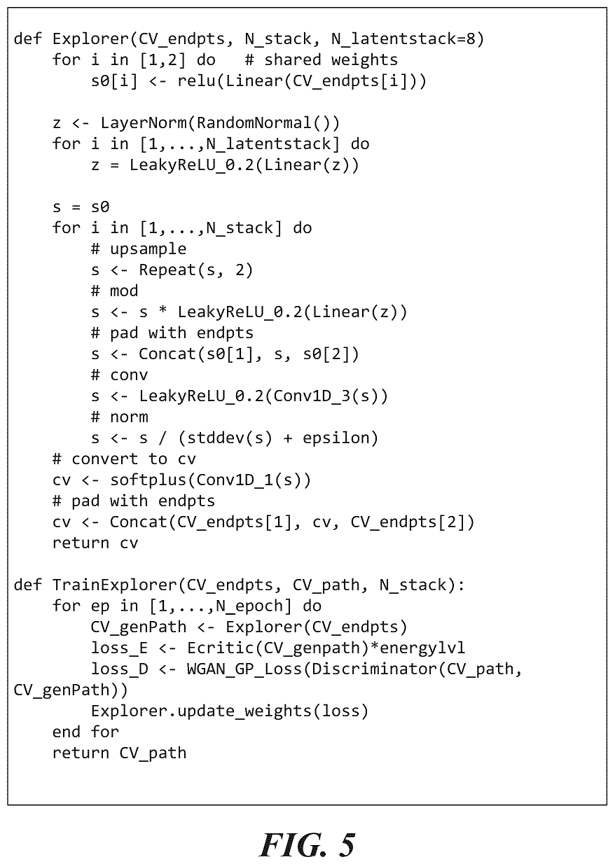

Referring now to FIG. 4, the Explorer further includes a generator and critics. The generator of Explorer applies the adapted progressive GAN by first generating only four structures along the path and gradually increasing the path resolution to include up to N structures, as specified by the user. An example algorithm is provided in FIG. 5.

The inputs of the Explorer's generator are the low-dimension representation of the first state and the second state, which are expected to remain the same throughout the training and runtime, and an array of random numbers generated by RandomNormal( ) as stated in FIG. 5. The output is N conformations in their low-dimension representation, collectively forming the path connecting the first state and the second state.

Primarily, Explorer's generator is trained to lower the penalty from the two critics, L_{Ecritic} and L_{discrimin}, which are part of the loss function of the Explorer's generator (Eq. 1). Three additional math-based critics contribute to the loss function: L_{diff} (Eq. 2) to maintain the proximity of neighboring structures along the path, L_{clash} (Eq. 3) to prevent residue-residue distances becoming too small, and Lᵥₐᵣ to control the diversity of the paths (Eq. 4).

$${L}_{generator} = {L}_{{E}_{c r i t i c}} + {L}_{d i s c r i m} + {L}_{diff} + {L}_{c l a s h} + {L}_{v a r} \qquad ( 1 )$$

$${L}_{diff} = {{mean}_{i ∈ ( 2 , N )} ( \frac{{‖ {cv}_{i} - c {v}_{i - 1} ‖}_{2}}{{‖ {cv}_{N} - c {v}_{1} ‖}_{2} / ( N - 1 )} - 1 )}^{2} \qquad ( 2 )$$

$${L}_{c l a s h} = {Σ}_{i ∈ ( 2 , N )} max ( 0.35 - {cv}_{i} , 0 ) \qquad ( 3 )$$

$${L}_{v a r} = {mean}_{i ∈ ( 2 , N )} max ( 0.05 - ‖ {CV}_{a} - C {V}_{b} ‖ , 0 ) \qquad ( 4 )$$

As a GAN, the Explorer also contains adversarial networks that serve as critics to provide feedback to the generator. In this implementation, two separate networks are used to serve as the critics, namely WGAN (Wasserstein GAN) and energy critics. Both networks score the conformations along the path individually, while additional math-based critics are deployed to enforce proximity between neighboring conformations along the path.

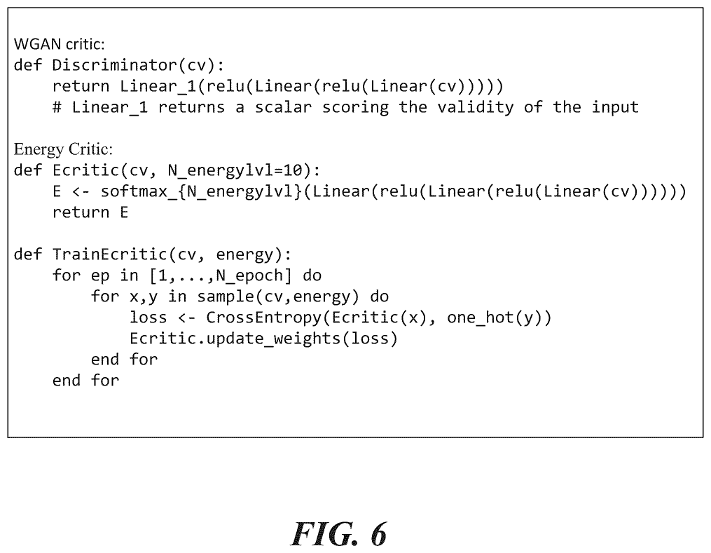

The WGAN critic (Arjovsky, M., Chintala, S., & Bottou, L. (2017), International conference on machine learning, PMLR) rates how realistic each individual generated conformation is as compared to those from the structure database. In this implementation, the WGAN critic includes a multilayer perceptron (MLP). The energy critic predicts the potential energy of each individual generated conformation. In this implementation, the energy critic also includes an MLP. Example algorithms of the WGAN and energy critic networks are shown in FIG. 6. For both the WGAN and energy critics, the inputs are individual generated conformations in their low-dimension representation. The output for the WGAN critic is a scalar floating number indicating how realistic the input is, such that the lower the output, the more realistic. The output of energy critic is the probability of the conformation belonging to each discretized energy bin.

The WGAN critic is trained to maximize the penalty score to conformations generated by the generator while minimizing the penalty score of conformations from the database. It also has a penalty to minimize its own gradient, as shown in the WGAN critic network's loss function, Equation 5.

$${L}_{d i s c r i m} = D ( c {v}_{g e n e r a t e d} ) - D ( c {v}_{d a t a b a s e} ) + {λ ( {‖ {∇}_{CV} D ( cv ) ‖}_{2} - 1 )}^{2} \qquad ( 5 )$$

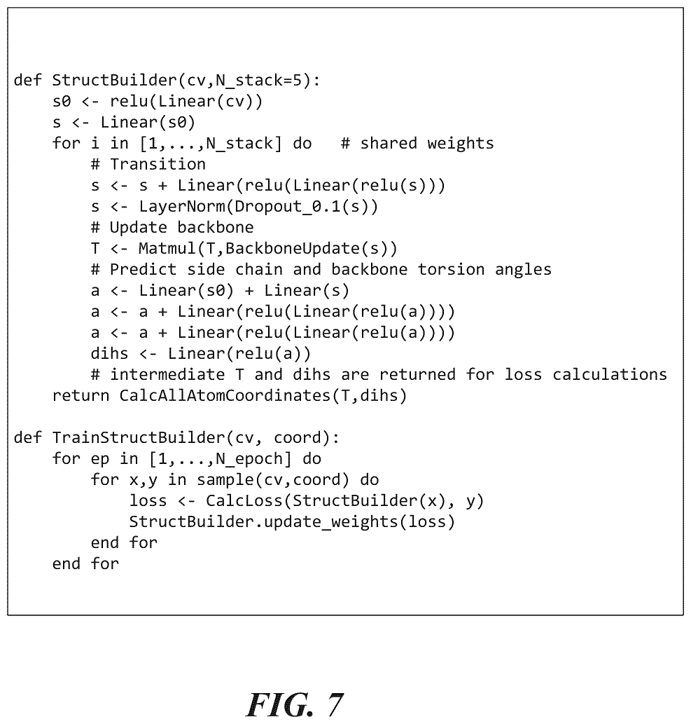

The Structure Builder module constructs the atomistic (all-atom) representation of a macromolecule conformation from its low-dimension representation. To achieve this, Structure Builder employs a neural network architecture that leverages residual connections and layer normalization. To lower the complexity of the problem, Structure Builder predicts the backbone orientation and dihedral angles then the cartesian coordinates of the atoms are computed from them. The network is stacked for N_stack times to gradually improve the output accuracy. An example algorithm of the Structure Builder is shown in FIG. 7.

Structure Builder takes each individual structures in low-dimension representation and converts them into array of 3D coordinates of each atom on the protein. The difference between the predicted and ground-truth atom positions under many different alignments are determined. Here, the difference of all intermediate backbone predictions from each stack of network and the final all-atom prediction are measured. The differences are summed together as the loss function for the Structure Builder. Additionally, the predicted dihedral angles are also compared to the ground-truth conformations using mean squared difference and added to the loss function of the Structure Builder.

To enable the system to explore a vast scale of macromolecule conformations, an active learning procedure is used where the system 100 interacts with an energy minimizer to optimize conformations and determine their ground-truth energies as described below:
i. Train Structure Builder and Explorer's energy critic using the first state input and the second state input.
ii. Explorer's generator and WGAN critic are trained to generate realistic conformations and transition paths.
iii. The system 100 generates plausible transition paths.
iv. Some output conformations are selected based on Explorer's critics and are optimized by energy minimization, which provides both the ground-truth conformation and respective energy.
v. Newly optimized conformations and their energies are used to retrain the Structure Builder and Explorer's energy critic.
vi. Repeat step ii-v.

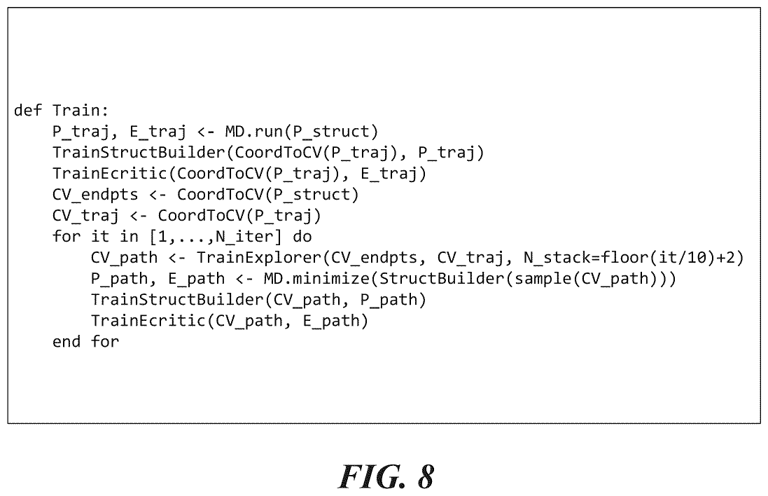

An example algorithm of the training procedure is shown in FIG. 8.

In some aspects, an array of random numbers is generated during each iterative run of training. For example, the array of random numbers may be generated using a random number generator and variations thereof. For example, an array of 64 random numbers, 128 random numbers, 256 random numbers, or increments thereof. After training, an array of random numbers is the only input to the system 100 to produce an ensemble of macromolecule conformations. It should be understood that the size of the array of random numbers given are for example only and are not limited to the provided example. It should be understood that the size of the array of random numbers is chosen to optimize performance of the method and system on the compute processor(s).

The low-dimension representation can be defined in any manner that fits the problem at hand, e.g. dihedral angles, contact distances, etc. In this implementation, carbon-alpha distances between selected protein residues were used.

After training, the system generates novel (not included in the input conformations) and realistic (having low energies) conformations of the input macromolecule, described by names and cartesian coordinates of all the atoms comprising the macromolecule. In this implementation, the output conformations are restricted to only those that form a continuous path between the two end-state inputs. Hence, the output is a path connecting the two end-state inputs, defined by a series of conformations along the transition path, with each conformation described by the cartesian coordinates of all atoms comprising the macromolecule. The number of conformations on each path is defined by the user. The path generated is unique between each run, changing according to the input random numbers.

The selected novel conformations are then provided to the energy minimizer module, which determines the local minimum energy of each conformation, thereby optimizing each conformation to its lowest energy state. The optimized conformations with energy labels are then stored in a structure database and energy database, respectively. The energy database and structure database are used to retrain the neural networks of the Explorer module and the Structure Builder module, respectively.

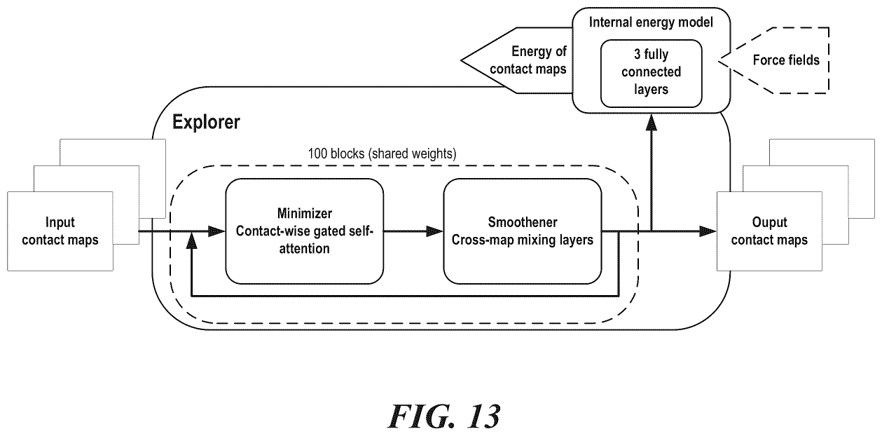

Aspects of the present disclosure can include different architectures for the Explorer module. FIG. 13 illustrates an embodiment including model-based reinforcement learning with a minimizer module that can lower the energies while maintaining relationships between different contacts and a “smoothener” module that can ensure adjacent maps are “adjacent.”

Alternatively, or additionally, the explorer can include a diffusion model trained on the contact maps for Gaussian-distributed latent representations of protein conformations. Rejection sampling can optionally be used to sample low energy pathways according to an energy critic. For example, the Explorer may be implemented using a different neural network, e.g. diffusion model, which does not distinguish between the first and second macromolecule states.

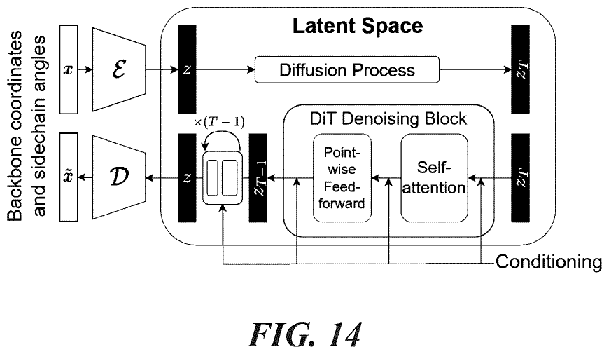

Referring now to FIG. 14, a Diffusion Transformer (DiT) may be used in the Explorer module. A DiT denoising module may be trained and used to repeatedly and gradually improve the output conformation quality. The loss function of the Explorer with DiT may include terms that minimize the energies as well as increasing variation of the generated conformations.

In some aspects, when only one macromolecule state is provided as input, the second macromolecule state will be generated by analyzing the sample conformations provided for the first macromolecule using mathematical methods (e.g. principal component analysis) and extrapolate to new conformations. Such conformations will be energy minimized and MC or MD simulations can be used to generate more conformations around the new conformation. Aspects of the present disclosure can include methods of training any/all of the models described herein.

Some embodiments of the present disclosure can include deep learning methods with a reinforcement learning model that can generate plausible transition pathways using only equilibrium simulations. Alternatively, or additionally, the deep learning models can be configured to recover features along the folding pathway and/or to construct a low-dimension reaction coordinate that can be used for enhanced sampling.

### Example: Biologic Structures

With reference to FIG. 1, an example embodiment of the disclosure system and method was designed and tested. The example embodiment includes a deep-learning method that learns to generate an initial guess of the transition pathway between two conformational states of a molecule from short equilibrium simulations and in the process, also provides an optimal set of collective variables to describe the pathway. The example embodiment can include two main modules: the Explorer and the Structure Builder (per FIG. 1). The Explorer uses a progressive, growing generative adversarial network (ProGAN) to construct a plausible transition pathway between the given end states in a reduced latent space, e.g., the residue-residue pairwise distances variable space in the case of a protein. The Structure Builder can build the all-atom structure of the molecule from the output of the Explorer. While the Structure Builder can be pre-trained using short trajectories, the Explorer can be trained with active learning. To obtain a low-energy pathway, the Explorer actively searches for new structures of lower energies, determined by the GAN discriminator and energy critic within the Explorer, demanding new training data on the fly, which will be provided by the molecular dynamics (MD) energy minimizer. The minimized structures can also be used to re-train the Structure Builder. After a path that satisfies the convergence criteria is generated, the weights of the Explorer energy critic will be analyzed to construct a set of the most relevant collective variables.

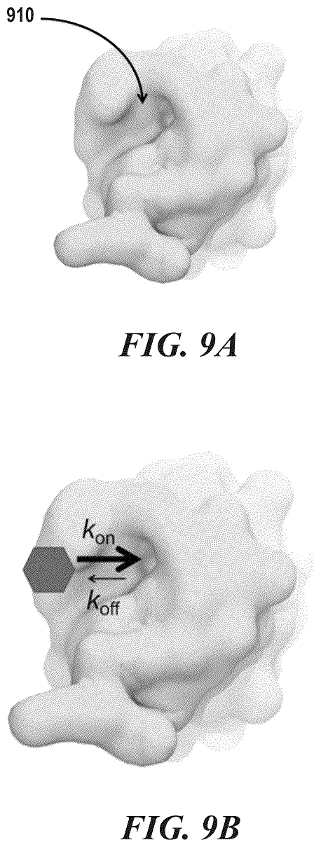

In some embodiments, the system can be used to generate a conformational ensemble from a single molecular structure. The ensemble can be designed to target certain properties. Non-limiting examples of properties that can be targeted include protein pocket volumes, such as “large pocket volume,” which can be used as features in a drug discovery process. An example pocket 910 is shown in FIG. 9A.

Additionally, embodiments of the present disclosure can be configured to identify drug binding/unbinding pathways, including optimization of binding rate (kₒₙ, large) and unbinding rate (k_{off}, small) that can improve drug efficacy, illustrated in FIG. 9B.

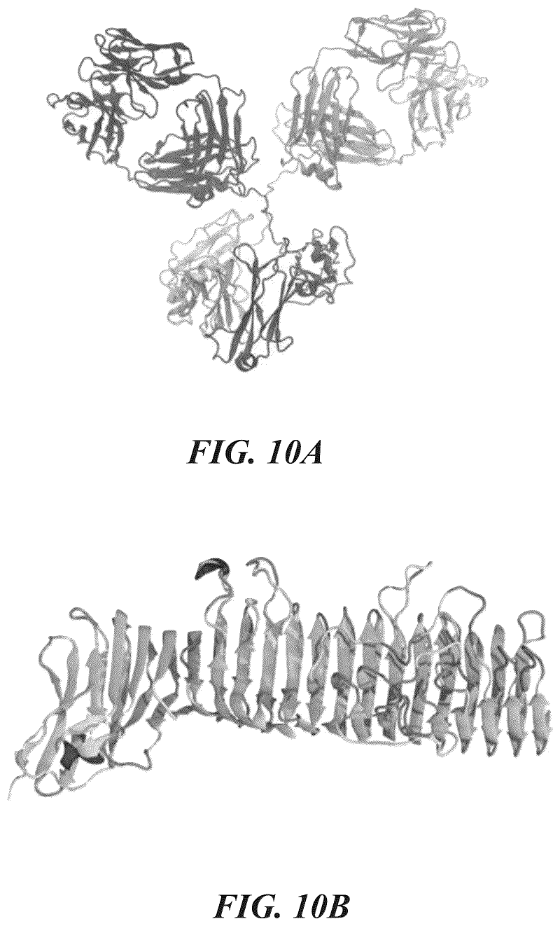

Additionally, embodiments of the present disclosure can predict conformational states of biologics. Biologics can include antibodies. Biologics are a class of drugs that can include proteins. FIG. 10A shows an example protein shape.

With reference to FIG. 10B, an example of a protein is shown. Embodiments of the present disclosure can simulate autotransporters like pertactin which can act as bacterial virulence factors. A major energy source for autotransporter passenger domain secretion can be its own folding. Embodiments of the present disclosure can perform enhanced sampling that can be used to simulate protein folding, including intermediate structures.

Additionally, embodiments of the present disclosure can predict conformational states of ribonucleic acid (RNA). FIG. 10C shows an example of a folded RNA molecule.

Additionally, embodiments of the present disclosure can predict conformational states of non-biological polymers. FIG. 10D shows an example of a folded polymer.

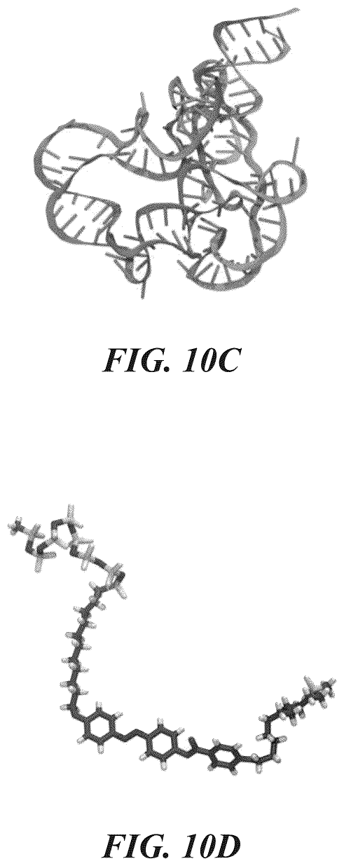

Additionally, embodiments of the present disclosure can predict interactions between two molecules. FIG. 11 shows an example of a polymer interacting with RNA via hydrogen bonds 1110 and hydrophobic interactions 1120.

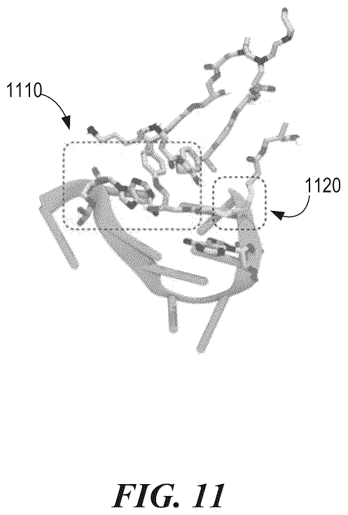

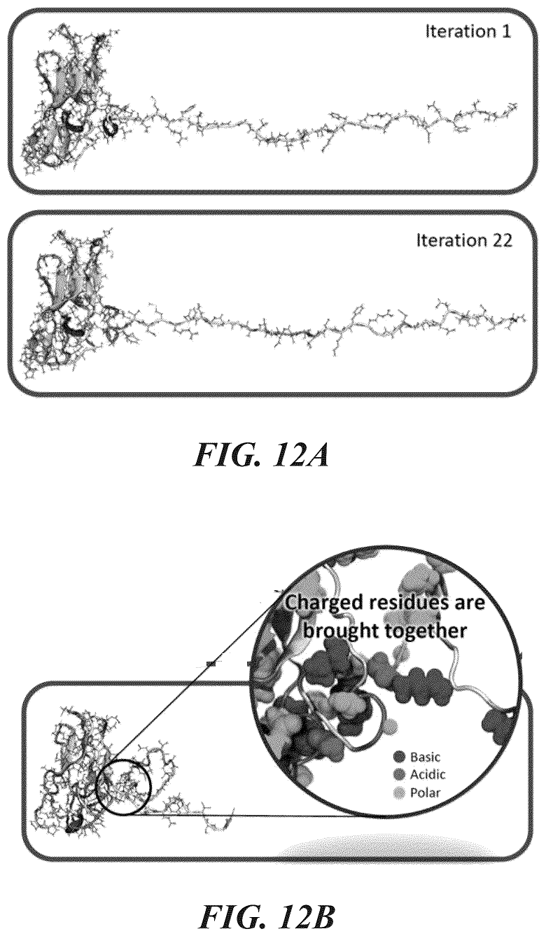

Embodiments of the present disclosure can output visualizations of folding pathways, as shown in FIGS. 12A and 12B.

### Example Operation

GAN can generate fake data via the generator, and the discriminator tries to determine if it's fake or real, and then stops. The exemplary GAN can generate real data on the fly and store it beyond the traditional stop point. In some embodiments, after the discriminator determines if the data is real or fake, part of the generated fake data, which might represent physically impossible structures, are passed to the energy minimizer to convert them to energetic favorable, aka physically possible, datapoints. The criteria for data selected for energy minimization depends on the specific implementation.

The energy minimizer is configured to expand the real dataset by generating new real datapoints. In conventional GAN training, the discriminator is repeatedly trained to classify data from some preselected dataset as real, while data from the generator as fake. In the exemplary GAN, this can be exactly the same as conventional GAN except the real dataset is expanding by data generated from the energy minimizer. This operation allows the exemplary GAN to generate data beyond the limitation of the preselected dataset. This is important because one of the usage or benefit is to find the path connecting two protein states, which can be assumed to only have training data on the two states and no data on the path. The expansion of the real dataset allows the newly generated datapoints on the path to not be discarded by the discriminator, but rather only discard data that are physically impossible (or energetically unfavorable).

An example training cycle can be summarized as follows:
(i) Generator of GAN generates data;
(ii) Discriminator of GAN judge whether the generated data is real or fake, and a separate neural network predicts the energy of the generated data;
(iii) Adjust the weights of the generator of GAN based on the discriminator of GAN and the energy predicting neural network
(iv) Adjust the weights of the discriminator of GAN to classify data from “real” dataset as real and data from generator of GAN as fake.
(v) Some generated data are passed to the energy minimizer. The energy minimizer may perform steepest decent on the datapoints and finds the nearest points from the input datapoints with lowest energy. The output from the energy minimizer may be saved as part of the “real” dataset. The energy minimizer also provides the correct energy label to these new “real” data.
(vi) Adjust the weights of the energy predicting neural network using the “real” dataset and its corresponding energy labels.
(vii) Repeat above operations.

Other machine learning and AI methods may be employed.

Machine Learning. In addition to the machine learning features described above, the system can be implemented using one or more artificial intelligence and machine learning operations. The term “artificial intelligence” can include any technique that enables one or more computing devices or comping systems (i.e., a machine) to mimic human intelligence. Artificial intelligence (AI) includes but is not limited to knowledge bases, machine learning, representation learning, and deep learning. The term “machine learning” is defined herein to be a subset of AI that enables a machine to acquire knowledge by extracting patterns from raw data. Machine learning techniques include, but are not limited to, logistic regression, support vector machines (SVMs), decision trees, Naïve Bayes classifiers, and artificial neural networks. The term “representation learning” is defined herein to be a subset of machine learning that enables a machine to automatically discover representations needed for feature detection, prediction, or classification from raw data. Representation learning techniques include, but are not limited to, autoencoders and embeddings. The term “deep learning” is defined herein to be a subset of machine learning that enables a machine to automatically discover representations needed for feature detection, prediction, classification, etc., using layers of processing. Deep learning techniques include but are not limited to artificial neural networks or multilayer perceptron (MLP).

Machine learning models include supervised, semi-supervised, and unsupervised learning models. In a supervised learning model, the model learns a function that maps an input (also known as feature or features) to an output (also known as target) during training with a labeled data set (or dataset). In an unsupervised learning model, the algorithm discovers patterns among data. In a semi-supervised model, the model learns a function that maps an input (also known as feature or features) to an output (also known as a target) during training with both labeled and unlabeled data.

Neural Networks. An artificial neural network (ANN) is a computing system including a plurality of interconnected neurons (e.g., also referred to as “nodes”). This disclosure contemplates that the nodes can be implemented using a computing device (e.g., a processing unit and memory as described herein). The nodes can be arranged in a plurality of layers such as input layer, an output layer, and optionally one or more hidden layers with different activation functions. An ANN having hidden layers can be referred to as a deep neural network or multilayer perceptron (MLP). Each node is connected to one or more other nodes in the ANN. For example, each layer is made of a plurality of nodes, where each node is connected to all nodes in the previous layer. The nodes in a given layer are not interconnected with one another, i.e., the nodes in a given layer function independently of one another. As used herein, nodes in the input layer receive data from outside of the ANN, nodes in the hidden layer(s) modify the data between the input and output layers, and nodes in the output layer provide the results. Each node is configured to receive an input, implement an activation function (e.g., binary step, linear, sigmoid, tanh, or rectified linear unit (ReLU) function), and provide an output in accordance with the activation function. Additionally, each node is associated with a respective weight. ANNs are trained with a dataset to maximize or minimize an objective function. In some implementations, the objective function is a cost function, which is a measure of the ANN's performance (e.g., error such as L1 or L2 loss) during training, and the training algorithm tunes the node weights and/or bias to minimize the cost function. This disclosure contemplates that any algorithm that finds the maximum or minimum of the objective function can be used for training the ANN. Training algorithms for ANNs include but are not limited to backpropagation. It should be understood that an artificial neural network is provided only as an example machine learning model. This disclosure contemplates that the machine learning model can be any supervised learning model, semi-supervised learning model, or unsupervised learning model. Optionally, the machine learning model is a deep learning model. Machine learning models are known in the art and are therefore not described in further detail herein.

A convolutional neural network (CNN) is a type of deep neural network that has been applied, for example, to image analysis applications. Unlike traditional neural networks, each layer in a CNN has a plurality of nodes arranged in three dimensions (width, height, depth). CNNs can include different types of layers, e.g., convolutional, pooling, and fully connected (also referred to herein as “dense”) layers. A convolutional layer includes a set of filters and performs the bulk of the computations. A pooling layer is optionally inserted between convolutional layers to reduce the computational power and/or control overfitting (e.g., by down sampling). A fully connected layer includes neurons, where each neuron is connected to all of the neurons in the previous layer. The layers are stacked similar to traditional neural networks. GCNNs are CNNs that have been adapted to work on structured datasets such as graphs.

Other Supervised Learning Models. A logistic regression (LR) classifier is a supervised classification model that uses the logistic function to predict the probability of a target, which can be used for classification. LR classifiers are trained with a data set (also referred to herein as a “dataset”) to maximize or minimize an objective function, for example, a measure of the LR classifier's performance (e.g., error such as L1 or L2 loss), during training. This disclosure contemplates that any algorithm that finds the minimum of the cost function can be used. LR classifiers are known in the art and are therefore not described in further detail herein.

A Naïve Bayes' (NB) classifier is a supervised classification model that is based on Bayes' Theorem, which assumes independence among features (i.e., the presence of one feature in a class is unrelated to the presence of any other features). NB classifiers are trained with a data set by computing the conditional probability distribution of each feature given a label and applying Bayes' Theorem to compute the conditional probability distribution of a label given an observation. NB classifiers are known in the art and are therefore not described in further detail herein.

A k-NN classifier is an unsupervised classification model that classifies new data points based on similarity measures (e.g., distance functions). The k-NN classifiers are trained with a data set (also referred to herein as a “dataset”) to maximize or minimize a measure of the k-NN classifier's performance during training. This disclosure contemplates any algorithm that finds the maximum or minimum. The k-NN classifiers are known in the art and are therefore not described in further detail herein.

A majority voting ensemble is a meta-classifier that combines a plurality of machine learning classifiers for classification via majority voting. In other words, the majority voting ensemble's final prediction (e.g., class label) is the one predicted most frequently by the member classification models. The majority voting ensembles are known in the art and are therefore not described in further detail herein.

A diffusion model is a type of generative model and is developed by unsupervised training. Diffusion models generate data by transforming simple distributions into more complex distributions of interest using stochastic process operations. Diffusion models have been used for generating new images, searching images, transforming text to images, and reverse image searches.

### Example Computing Device

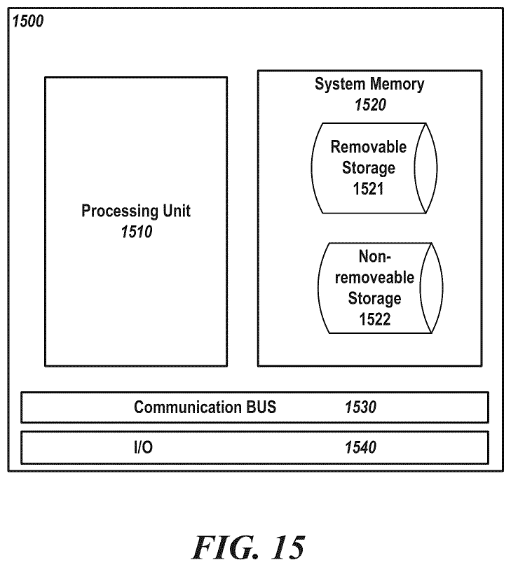

Referring to FIG. 15, an example computing device 1500 upon which the methods described herein may be implemented is illustrated. It should be understood that the example computing device 1500 is only one example of a suitable computing environment upon which the methods described herein may be implemented. Optionally, the computing device 1500 can be a well-known computing system including, but not limited to, personal computers, servers, handheld or laptop devices, multiprocessor systems, microprocessor-based systems, network personal computers (PCs), minicomputers, mainframe computers, embedded systems, and/or distributed computing environments including a plurality of any of the above systems or devices. Distributed computing environments enable remote computing devices, which are connected to a communication network or other data transmission medium, to perform various tasks. In the distributed computing environment, the program modules, applications, and other data may be stored on local and/or remote computer storage media.

In its most basic configuration, computing device 1500 typically includes at least one processing unit 1510 and system memory 1520. Depending on the exact configuration and type of computing device, system memory 1510 may be volatile (such as random access memory (RAM), non-volatile (such as read-only memory (ROM), flash memory, etc.), or some combination of the two. The processing unit 1510 may be a standard programmable processor that performs arithmetic and logic operations necessary for the operation of the computing device 1500. The computing device 1500 may also include a communication bus 1530 or other communication mechanism for communicating information among various components of the computing device 1500.

Computing device 1500 may have additional features/functionality. For example, computing device 1500 may include additional storage such as removable storage 1521 and non-removable storage 1522, including, but not limited to, magnetic or optical disks or tapes. Computing device 1500 may also contain network connection(s) that allow the device to communicate with other devices. Computing device 1500 may also have input and output means 1540 such as a keyboard, mouse, touch screen, a display, speakers, printer, etc. The additional devices may be connected to the communication bus 1530 in order to facilitate the communication of data among the components of the computing device 1500. All these devices are well-known in the art and need not be discussed at length here.

The processing unit 1510 may be configured to execute program code encoded in tangible, computer-readable media. Tangible, computer-readable media refers to any media that is capable of providing data that causes the computing device 1500 (i.e., a machine) to operate in a particular fashion. Various computer-readable media may be utilized to provide instructions to the processing unit 1510 for execution. Example of tangible, computer-readable media may include, but is not limited to, volatile media, non-volatile media, removable media, and non-removable media implemented in any method or technology for storage of information such as computer-readable instructions, data structures, program modules or other data. System memory 1520, removable storage 1521, and non-removable storage 1522 are all examples of tangible, computer storage media. Examples of tangible, computer-readable recording media include, but are not limited to, an integrated circuit (e.g., field-programmable gate array or application-specific IC), a hard disk, an optical disk, a magneto-optical disk, a floppy disk, a magnetic tape, a holographic storage medium, a solid-state device, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices.

In an example implementation, the processing unit 1510 may execute program code stored in the system memory 1520. For example, the communication bus 1530 may carry data to the system memory 1520, from which the processing unit 1510 receives and executes instructions. The data received by the system memory 1520 may optionally be stored on the removable storage 1521 or the non-removable storage 1522 before or after execution by the processing unit 1510.

It should be understood that the various techniques described herein may be implemented in connection with hardware or software or, where appropriate, with a combination thereof. Thus, the methods and apparatuses of the presently disclosed subject matter, or certain aspects or portions thereof, may take the form of program code (i.e., instructions) embodied in tangible media, such as floppy diskettes, CD-ROMs, hard drives, or any other machine-readable storage medium where, when the program code is loaded into and executed by a machine, such as a computing device, the machine becomes an apparatus for practicing the presently disclosed subject matter. In the case of program code execution on programmable computers, the computing device generally includes a processor, a storage medium readable by the processor (including volatile and non-volatile memory and/or storage elements), at least one input device, and at least one output device. One or more programs may implement or utilize the processes described in connection with the presently disclosed subject matter, e.g., through the use of an application programming interface (API), reusable controls, or the like. Such programs may be implemented in a high-level procedural or object-oriented programming language to communicate with a computer system. However, the program(s) can be implemented in assembly or machine language, if desired. In any case, the language may be a compiled or interpreted language, and it may be combined with hardware implementations.

### General Definitions

As used in the description and the appended claims, the singular forms “a,” an, and “the” include plural referents unless the context clearly dictates otherwise. Thus, for example, reference to “a composition” includes mixtures of two or more such compositions, reference to “an agent” includes mixtures of two or more such agents, reference to “the component” includes mixtures of two or more such components, and the like.

By “comprising” or “containing” or “including” is meant that at least the name compound, element, particle, or method step is present in the composition or article or method, but does not exclude the presence of other compounds, materials, particles, method steps, even if the other such compounds, material, particles, method steps have the same function as what is named.

“Optional” or “optionally” means that the subsequently described event or circumstance can or cannot occur, and that the description includes instances where the event or circumstance occurs and instances where it does not.

Ranges can be expressed herein as from “about” one particular value, and/or to “about” another particular value. By “about” is meant within 5% of the value, e.g., within 4, 3, 2, or 1% of the value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

“Exemplary” means “an example of” and is not intended to convey an indication of a preferred or ideal embodiment. “Such as” is not used in a restrictive sense, but for explanatory purposes.

Values can be expressed herein as an “average” value. “Average” generally refers to the statistical mean value.

By “substantially” is meant within 5%, e.g., within 4%, 3%, 2%, or 1%.

“Exemplary” means “an example of” and is not intended to convey an indication of a preferred or ideal embodiment. “Such as” is not used in a restrictive sense, but for explanatory purposes.

It is understood that throughout this specification the identifiers “first” and “second” are used solely to aid in distinguishing the various components and operations of the disclosed subject matter. The identifiers “first” and “second” are not intended to imply any particular order, amount, preference, or importance to the components or operations modified by these terms.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, by a “subject” is meant an individual. Thus, the “subject” can include domesticated animals (e.g., cats, dogs, etc.), livestock (e.g., cattle, horses, pigs, sheep, goats, etc.), laboratory animals (e.g., mouse, rabbit, rat, guinea pig, etc.), and birds. “Subject” can also include a mammal, such as a primate or a human. Thus, the subject can be a human or veterinary patient. The term “patient” refers to a subject under the treatment of a clinician, e.g., physician.

The term “inhibit” refers to a decrease in an activity, response, condition, disease, or other biological parameter. This can include but is not limited to the complete ablation of the activity, response, condition, or disease. This can also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed. For example, the terms “prevent” or “suppress” can refer to a treatment that forestalls or slows the onset of a disease or condition or reduced the severity of the disease or condition. Thus, if a treatment can treat a disease in a subject having symptoms of the disease, it can also prevent or suppress that disease in a subject who has yet to suffer some or all of the symptoms.

Disclosed herein are variants of naturally occurring proteins. Substitutional or replacement variants typically contain the exchange of one amino acid for another at one or more sites within the protein and may be designed to modulate one or more properties of the polypeptide, particularly to reduce its immunogenicity/antigenicity, reduce any side effects in a subject, or increase its efficacy. Substitutions of this kind preferably are conservative, that is, one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine. An antigenic region of a polypeptide may be substituted for a less antigenic region; the less antigenic region may contain residues that are identical to the corresponding residues in the native protein, yet also contain some conservative substitutions and/or nonconservative substitutions.

In addition to a deletion or substitution, a modified protein may possess an insertion of residues, which typically involves the addition of at least one residue in the polypeptide. This may include the insertion of a targeting peptide or polypeptide or simply a single residue. Terminal additions, called fusion proteins, are discussed below.

The term “biologically functional equivalent” (also referred to herein as a “functional derivative, fragment, or variant thereof”) is well understood in the art and is further defined in detail herein. Accordingly, sequences that have between about 70% and about 80%, or between about 81% and about 90%, or even between about 91% and about 99% of amino acids that are identical or functionally equivalent to the amino acids of a native polypeptide are included, provided the biological activity of the protein is maintained. A modified protein may be biologically functionally equivalent to its native counterpart.

It also will be understood that amino acid and nucleic acid sequences may include additional residues, such as additional N- or C-terminal amino acids or 5′ or 3′ sequences, and yet still be essentially as set forth in one of the sequences disclosed herein, so long as the sequence meets the criteria set forth above, including the maintenance of biological protein activity where protein expression is concerned. The addition of terminal sequences particularly applies to nucleic acid sequences that may, for example, include various non-coding sequences flanking either of the 5′ or 3′ portions of the coding region or may include various internal sequences, i.e., introns, which are known to occur within genes.

The following is a discussion based upon changing of the amino acids of a protein to create an equivalent, or even an improved, modified molecule. For example, certain amino acids may be substituted for other amino acids in a protein structure without appreciable loss of interactive binding capacity with structures such as, for example, binding sites to substrate molecules. Since it is the interactive capacity and nature of a protein that defines that protein's biological functional activity, certain amino acid substitutions can be made in a protein sequence, and in its underlying DNA coding sequence, and nevertheless produce a protein with like properties. It is thus contemplated that various changes may be made in the DNA sequences of genes without appreciable loss of their biological utility or activity, as discussed below.

A proteinaceous molecule has “homology” or is considered “homologous” to a second proteinaceous molecule if one of the following “homology criteria” is met: 1) at least 30% of the proteinaceous molecule has sequence identity at the same positions with the second proteinaceous molecule; 2) there is some sequence identity at the same positions with the second proteinaceous molecule and at the nonidentical residues, at least 30% of them are conservative differences, as described herein, with respect to the second proteinaceous molecule; or 3) at least 30% of the proteinaceous molecule has sequence identity with the second proteinaceous molecule, but with possible gaps of nonidentical residues between identical residues. As used herein, the term “homologous” may equally apply to a region of a proteinaceous molecule, instead of the entire molecule. If the term “homology” or “homologous” is qualified by a number, for example, “50% homology” or “50% homologous,” then the homology criteria, with respect to 1), 2), and 3), is adjusted from “at least 30%” to “at least 50%.” Thus, it is contemplated that there may.

Although example embodiments of the present disclosure are explained in some instances in detail herein, it is to be understood that other embodiments are contemplated. Accordingly, it is not intended that the present disclosure be limited in its scope to the details of construction and arrangement of components set forth in the following description or illustrated in the drawings. The present disclosure is capable of other embodiments and of being practiced or carried out in various ways.

In describing example embodiments, terminology will be resorted to for the sake of clarity. It is intended that each term contemplates its broadest meaning as understood by those skilled in the art and includes all technical equivalents that operate in a similar manner to accomplish a similar purpose. It is also to be understood that the mention of one or more steps of a method does not preclude the presence of additional method steps or intervening method steps between those steps expressly identified. Steps of a method may be performed in a different order than those described herein without departing from the scope of the present disclosure. Similarly, it is also to be understood that the mention of one or more components in a device or system does not preclude the presence of additional components or intervening components between those components expressly identified.

### EXEMPLARY ASPECTS

Clause 1. A system for generating a candidate molecular structure, comprising: at least one processor; a memory operably coupled to the at least one processor, the memory having computer-executable instructions stored thereon; a molecular conformational change module stored in the memory that, when executed by the at least one processor, is configured to: receive a source molecular model or a contact map thereof (e.g., of end-point structures of a source protein; can be other latent or functional space); determine, using a trained deep learning neural network, one or more candidate paths in the contact map from one or more determined hypothetical paths, wherein the deep learning neural network (e.g., generative adversarial network) is trained using source protein model or contact map to generate a plausible transition pathway for a molecular structure by scoring hypothetical paths in the contact map using a physics-based model (e.g., energy model); and determine one or more candidate protein structures, including a first candidate protein structure, by predicting an atom structure model determined using the one or more candidate paths in the contact map, wherein each of the one or more candidate molecular structures has at least one conformational ensemble; wherein the first candidate molecular structure is subsequently synthetized and/or simulated to evaluate for one or more target properties at the at least one conformational ensemble of the first candidate molecular structure; and perform energy minimization using molecular dynamics engine on one or more generated structures and use the newly minimized structures and their corresponding energies as new training data, and the whole model can be iterated until the results reach satisfactory.
Clause 2. The system of clause 1, wherein the trained deep learning network comprises a generative adversarial network or diffusion model or one or more autoencoders.
Clause 3. The system of clause 1, wherein the physics-based model comprises a molecular dynamics engine.
Clause 4. The system of clause 3, wherein the molecular dynamics engine comprises force field based analysis.
Clause 5. The system of clause 1, wherein the trained deep learning neural network comprises a first neural network.
Clause 6. The system of clause 5, wherein the first neural network is configured to construct a path iteratively as contact maps, which in some embodiments can be Ca pairwise distance space.
Clause 7. The system of clause 1, wherein the operation to determine one or more candidate molecular structures is performed with a neural network configured to convert the path into an all-atom structure.
Clause 8. The system of clause 5, wherein the first neural network is trained using the physics-based model.
Clause 9. The system of clause 7, wherein the neural network is trained using the physics-based model.
Clause 10. The system of clause 5, wherein the first neural network is trained using reinforcement learning.
Clause 11. The system of any one of clauses 1-10, wherein the trained deep learning network comprises a generative adversarial network (GAN) configured, in an iterative manner, to generate the plausible transition pathway for the molecular structure via the trained deep learning neural network as a generator, wherein the GAN comprises a discriminator configured to determine if the plausible transition pathway for the molecular structure represents a physically impossible structure, and wherein that plausible transition pathway for the molecular structure is then converted by the energy minimization (e.g., energy minimizer) and energy model to a physically possible structure.
Clause 12. The system of clause 11, wherein the energy minimization (e.g., energy minimizer) is configured to generate new real datapoints (i.e., physically possible structures) that is stored and additionally used (e.g., in a secondary training loop) as a source structure for the training.
Clause 13. The system of clause 11, wherein training of the generative adversarial network comprises: (i) generator of GAN generating data; (ii) discriminator of GAN determining whether the generated data is real or fake, and a separate neural network predicts the energy of the generated data; (iii) adjusting the weights of the generator of GAN based on the discriminator of GAN and the energy predicting neural network; (iv) adjusting the weights of the discriminator of GAN to classify data from “real” dataset as real and data from generator of GAN as fake; (v) passing, at least one or some generated data, to an energy minimizer (e.g., wherein the energy minimizer is configured to perform steepest decent on the datapoints and finds the nearest points from the input datapoints with lowest energy, wherein the energy minimizer is saved as part of the “real” dataset, and wherein the energy minimizer also provides the correct energy label to these new “real” data); (vi) adjusting the weights of the energy predicting neural network using the “real” dataset and its corresponding energy labels; and (vii) repeat above steps i-vii in the iterative manner.
Clause 14. A non-naturally occurring molecular structure identified using the system of any one of clauses 1-13.
Clause 15. A method of treating a subject, the method comprising administering to the subject molecule of clause 1-13.

What is claimed is:

1. A method for generating a candidate macromolecule structure having one or more target properties, the method comprising:
receiving a first input and a second input, wherein the first input and the second input are related to a first macromolecule state and a second macromolecule state, respectively;
training a first neural network on respective energies of the first macromolecule state and the second macromolecule state and energies from an energy database;
generating a plurality of macromolecule conformations in a low-dimension representation using the trained first neural network initiated with a set of random numbers;
determining respective energy of each macromolecule conformation of the plurality of macromolecule conformations;
selecting a set of candidate macromolecule conformations from the plurality of macromolecule conformations, wherein the set of candidate macromolecule conformations are selected based on one or more scores related to the first macromolecule state, the second macromolecule state, and the energy database;
training a second neural network on macromolecule conformations of the first macromolecule state and the second macromolecule state and macromolecule conformations from a structure database, wherein the structure database comprises conformations in all-atom representation and related low-dimension representation;
determining, for each macromolecule conformation of the set of candidate macromolecule conformations, an all-atom representation from a low-dimension representation using the trained second neural network;
determining a local minimum energy of each candidate macromolecule conformation of the set of candidate macromolecule conformations to generate a set of optimized candidate macromolecule conformations and related energies;
adding the optimized candidate macromolecule conformations to the structure database and related energies to the energy database;
outputting one or more conformations of the set of optimized candidate macromolecule conformations, wherein the one or more conformations of the set of optimized candidate macromolecule conformations are subsequently synthetized and/or simulated to evaluate for one or more target properties; and
repeating the operations until a threshold associated the one or more target properties is reached or for a pre-determined number of iterations.

2. The method of claim 1, wherein the first macromolecule state comprises a plurality of macromolecule conformations, and the second macromolecule state comprises a plurality of macromolecule conformations.

3. The method of claim 1, wherein the first macromolecule state comprises a single macromolecule conformation, and the second macromolecule state comprises a single macromolecule conformation.

4. The method of claim 3, further comprise sampling the first macromolecule state and the second macromolecule state in equilibrium conditions using statistical simulations or physics-based simulations, wherein the sampled equilibrium conformations and related energies of the first macromolecule state and second macromolecule state are used for training the first neural network and the second neural network.

5. The method of claim 1, further comprising determining respective energies of the first macromolecule state of the first input state and determining respective energies of the second macromolecule state of the second input state.

6. The method of claim 1, wherein training the first neural network and the second neural network comprises reinforcement learning operations.

7. The method of claim 1, wherein the first neural network is a deep learning neural network.

8. The method of claim 7, wherein the first neural network comprises a generative adversarial network or diffusion model or one or more autoencoders.

9. The method of claim 8, wherein the first neural network comprises a generative adversarial network (GAN) configured, in an iterative manner, to generate a plausible low-energy pathway between the first macromolecule state and the second macromolecule state, wherein the GAN comprises a discriminator configured to determine if the plausible low-energy pathway between the first macromolecule state and the second macromolecule state comprises physically possible macromolecule conformations.

10. The method of claim 9, wherein training of the generative adversarial network comprises:
i. generating macromolecule conformations at a generator of the GAN;
ii. determining related energies of the generated macromolecule conformations at an energy predicting neural network;
iii. scoring related energies of the generated macromolecule conformations relative to the energy database at a discriminator of GAN;
iv. adjusting a weights of the generator of GAN based on the discriminator of GAN and the energy predicting neural network;
v. adjusting the weights of the discriminator of GAN to classify data from “real” dataset as real and data from generator of GAN as “fake”;
vi. passing, at least one generated macromolecule conformation, to an energy minimizer, wherein the energy minimizer is configured to determine a local minimum of an energy function describing the at least one generated macromolecule conformation, and wherein the energy minimizer is saved as part of the “real” dataset;
vii. adjusting the weights of the energy predicting neural network using the “real” dataset and its corresponding energy labels; and
viii. repeat above operations i-vii in an iterative manner.

11. The method of claim 1, wherein determining, for each macromolecule conformation of the set of candidate macromolecule conformations, an all-atom representation from a low-dimension representation using the trained second neural network, comprises:
predicting a backbone orientation;
scoring the predicted backbone orientation relative to the conformations of the structure database;
predicting dihedral angles;
scoring the predicted dihedral angles relative to the conformations of the structure database;
adding the backbone orientation score and dihedral angles score to a loss function of the trained second neural network;
determining a coordinates of all atoms relative to the scored backbone orientation and scored dihedral angles using the trained second neural network.

12. The method of claim 1, wherein determining respective energies comprises evaluating a force field energy function.

13. The method of claim 1, wherein the first state input and the second state input comprise all-atom representations of macromolecule conformations.

14. The method of claim 13, further comprising converting the all-atom representations of macromolecule confirmation of the first state input and the second state input to low-dimension representations before training the first neural network.

15. The method of claim 1, wherein the first state input and the second state input comprise low-dimension representations of macromolecule conformations.

16. The method of claim 1, wherein the low-dimension representation is defined by dihedral angles, contact distances, or distances between selected macromolecule residues.

17. The method of claim 16, wherein the low-dimension representation is defined by Ca pairwise distance space.

18. The method of claim 1, wherein the selected set of candidate structures comprises a number of candidate structures wherein the number is defined by a user input.

19. The method of claim 1, wherein the set of candidate structures are equally spaced along a transition path between the first macromolecule state and the second macromolecule state.

20. The method of claim 1, comprising receiving a first input and a second input, wherein the first input is related to a first macromolecule state, and further comprising generating a second macromolecule state.

21. A system for generating a candidate macromolecule structure having one or more target properties, comprising:
at least one processor;
a memory operably coupled to the at least one processor, the memory having computer-executable instructions stored thereon, that when executed by the at least one processor, causes the processor to:
receive a first input and a second input, wherein the first input and the second input are related to a first macromolecule state and a second macromolecule state, respectively;
determine respective energies of the first macromolecule state and the second macromolecule state;
train a first neural network on respective energies of the first macromolecule state and the second macromolecule state and energies from an energy database;
generate a plurality of macromolecule conformations in a low-dimension representation using the trained first neural network initiated with a set of random numbers;
determine respective energy of each macromolecule conformation of the plurality of macromolecule conformations;
select a set of candidate macromolecule conformations from the plurality of macromolecule conformations, wherein the set of candidate macromolecule conformations are selected based on one or more scores related to the first macromolecule state, the second macromolecule state, and the energy database;
train a second neural network on macromolecule conformations of the first macromolecule state and the second macromolecule state and macromolecule conformations from a structure database, wherein the structure database comprises conformations in all-atom representation and related low-dimension representation;
determine, for each macromolecule conformation of the set of candidate macromolecule conformations, an all-atom representation from a low-dimension representation using the trained second neural network;
determine a local minimum energy of each candidate macromolecule conformation of the set of candidate macromolecule conformations to generate a set of optimized candidate macromolecule conformations and related energies;
add the optimized candidate macromolecule conformations to the structure database and related energies to the energy database;
output one or more of the set of optimized candidate macromolecule conformations, wherein the one or more conformations of the set of optimized candidate macromolecule conformations, wherein the one or more conformations of the set of optimized candidate macromolecule conformations are subsequently synthetized and/or simulated to evaluate for one or more target properties; and
repeat the operations until a threshold associated the one or more target properties is reached or for a pre-determined number of iterations.
